(19) United States
(12) Patent Application Publication
(10) Pub. No.: US 2005/0033390 A1
(43) Pub. Date: Feb. 10, 2005

(54) Thermal joint and bone compression system

(76) Inventor: Dean Edward McConnell, Fishers, IN (US)

Correspondence Address: Dean E. McConnell, 9609 Conifer Court, Fishers, IN, 46038, US

(21) Appl. No.: 10/631,188

(22) Filed: Jul. 31, 2003

(51) Int. Cl. A61F 7/00

(52) U.S. Cl. 607/104; 607/108

(57) ABSTRACT

A thermal compression system adapted to encompass a knee or any other portion of a limb is disclosed. The thermal compression system includes a container that has a fluid output line and a fluid input line. A fluid pump is connected with the fluid output line of the container and a fluid input of a cuff. The cuff is designed to be attached to an area of the body being treated. The pump fills the cuff with fluid and the fluid may exit the cuff through a fluid output of the cuff that is connected with the fluid input line of the container.

### FIELD OF THE INVENTION

The present invention relates generally to systems and methods used in the application of fluid in combination with compression to certain injured portions of the human body and more particularly, to a system and method for therapeutically applying fluid in combination with compression to a portion of a limb and replacing the fluid in the cuff applied to the limb.

### BACKGROUND OF THE INVENTION

The therapeutic value of simultaneous application of cold and compression to an injured body part is widely accepted in the medical community, and the acronym RICE for Rest, Ice, Compression, and Elevation, for the primary treatment of injury to joints and limbs of the body, is practiced routinely. For example, after knee surgery, compression and cold are almost universally applied to control the swelling and the commonly occurring hemarthrosis that causes pain and delays rehabilitation

Methods used for postoperative cold and compression traditionally have been applied separately—compression most commonly by an elastic bandage wrapped around the knee, and the cold by a superimposed plastic bag filled with ice. This approach is simple and economical, but it has complications. It has been demonstrated that an ace wrap applied to the knee at a moderate compression retard venous circulation and may contribute to thromboembolism. In addition, a wide compression variability exists in the application of an elastic wrap.

Several other types of devices have been developed for the application of cold and compression, and studies have demonstrated their relative effectiveness. Sloan, et al., in a 1988 study on “Effects of Cold and Compression on Edema”, reported in The Physician and Sports Medicine, Vol. 16, No. 8, August 1988, pp. 116-120, showed that a Cryopac sleeve that applied cold in a cuff inflated to 30 mm hg by Freon gas was highly effective in reducing edema. It was reported in 1989 in The American Journal of Sports Medicine, Vol. 17, No. 3, pp. 344-349, that using a Hot Ice Thermal Blanket machine to apply cold postoperatively to the knee reduced pain medication required by the patients.

A wide variety of systems have been advanced for the application of compression of cold. Some systems that have been developed include an elastic wrap, the simple ice bag, a freon inflated sleeve, ice cubes in a strapped-on blanket, and a gel insert in a strapped on sleeve. None of these systems or any other known system have addressed the need for maximizing the compression and cold in areas where needed, while minimizing compression in those areas most sensitive to restriction of venous circulation. In addition, a need exists for extending the time period and effort needed to keep the system cold that is being applied to the portion of the body being treated.

One known device that attempts to solve these problems is set forth in U.S. Pat. No. 5,314,455, issued to Johnson, Jr. et al. on May 24, 1994, which is hereby incorporated by reference in its entirety. This invention sets forth a cuff with a watertight cavity shaped to envelope only the anterior and sides of the knee, including particularly the suprapatellar pouch, by using a pair of distal arms. The cuff is held in place with an upper proximal strap and a lower distal strap that avoid the popliteal area and minimize constriction. When the cuff is applied to the knee, the straps are secured, but not tightened. Then a first amount of compression is applied to the knee by inflating the cuff to a predetermined amount, which causes the cavity to expand. The expansion tensions the straps and applies compression to the areas of the knee under the cavity.

While the device disclosed in the Johnson, Jr. et al. patent addresses some of the issues that need to be addressed in thermal compression devices, its design is lacking in several areas in which improvements could be made. The device does not cover the areas needed covered efficiently and tends to balloon up and not make contact as effectively as could be with the portion of the limb in all of the areas that are needed. As such, a cuff is needed that will apply cold to almost the entire knee, while minimizing pressure that is applied in areas where blood flow is important.

In addition, in order to fill the cuff with fluid, the patient using the system must manually raise the container that holds the fluid up above the cuff to a predetermined distance of height and allow gravity to fill the cuff. The patient must hold the container above the cuff until it is filled, which requires a lot of effort from the patient or assistance from someone helping the patient. For a patient that just had surgery and is in severe to moderate pain, this process can cause further pain and can be extremely frustrating.

After a relatively short period of time, the cold or hot fluid inside the cuff will become either hot or cold, depending on which type of fluid is being used, and will need to be replaced. In order to replace the fluid, the patient must position the container holding the fluid a predetermined distance lower than the cuff. This allows the fluid to be siphoned from the cuff back into the container. Again, this process requires the patient to move around, which may be uncomfortable for the patient or simply just downright frustrating. To that end, the patient must manually fill and empty the cuff using a physical effort.

As such, a need exists for a thermal compression system that can evenly apply cold or warmth to a desired area of a human body, avoids restricting blood flow in areas where circulation is needed, and does not require interaction by the patient once the cuff is placed in the location to be treated.

### SUMMARY OF THE INVENTION

A thermal compression system is disclosed that includes an electric fluid pump that is used to transfer fluid from a fluid container to a cuff that is attached to a portion of the body that is being treated. The fluid container includes a fluid output line and a fluid input line. The fluid output line of the fluid container may be connected with an input of the electric fluid pump. A pump fluid output line may be connected to a connection device that is connected with the cuff. The connection device may allow the pump fluid output line to be quickly and easily connected and disconnected from the cuff.

The cuff of the thermal compression system advantageously applies compression and thermal therapy to the portion of the body that is being treated. The cuff is mostly designed from a flexible fluid impervious material that is capable of holding fluid. The cuff includes a thermal section or chamber that receives fluid from the fluid pump that is in turn used to apply thermal therapy to the portion of the body being treated. The thermal section may cover the entire area of the area being treated or select portions of the area being treated. In addition, the thermal section applies a minimal amount of compression or pressure to the portion of the body that is being treated.

The cuff may also include a compression cavity that is used to apply compression or pressure to at least one portion of the body that is being treated. The compression cavity may be filled with fluid from the fluid pump or may be filled with air. The amount of pressure applied will vary depending upon the application that the thermal compression system is designed for use and the volume of fluid the compression cavity holds. The compression cavity is also formed in a manner that avoids applying pressure to areas that are being treated that may contain a blood flow path. As such, the compression cavity applies pressure in areas where it is needed and avoids applying pressure in areas where it will restrict blood flow in the blood flow paths of the portion of the body being treated.

The cuff may also include an output that is connected with the fluid input line of the fluid container. As such, fluid is capable of being pumped into the cuff and out of the cuff by the fluid pump. This eliminates the need for the patient to be concerned with manually replacing the fluid that applies thermal therapy. For example, the patient does not need to manually change ice or gel packs or manually lift the fluid container to a height above or below the cuff to fill and drain the cuff. Unnecessary movement that the patient may need to make to replace the fluid or ice packs is thereby eliminated because the present invention provides a hands free therapy system once the cuff is placed on the portion of the body to be treated and the fluid pump is turned on.

The thermal compression system may also include a control unit. The control unit may be connected with the fluid pump and may be used to control the overall operation of the fluid pump. The control unit may control the flow rate of fluid that the fluid pump transfers into the cuff over a period of time and may also control the manner in which the fluid pump runs. The control unit may run the fluid pump continuously or it may run the fluid pump for a predetermined amount of time. For example, the control unit may run the fluid pump for thirty-seconds every five minutes. The purpose of the fluid pump is to transfer fluid from the fluid container to the cuff so that the fluid in the cuff does not need to be replaced by any manual user interaction.

The thermal compression system may also include at least one temperature sensor that may be located in the cuff that monitors the temperature of the fluid in the cuff. If the temperature of the fluid in the cuff reaches a predetermined value, the control unit may turn the fluid pump on to supply the cuff with new fluid from the fluid container and direct the old fluid back to the fluid container so that it may be chilled to a lower temperature again for later use or reheated to a higher temperature for later use.

The thermal compression system may also include at least one pressure sensor that is located in the cuff. The pressure sensor may be located in the areas of the cuff that do not include the compression cavity. As such, the pressure sensor may monitor the pressure that is being applied to the portion of the body by the fluid section. If, for some reason, the pressure becomes to high, an indication may be given to the patient such as an alarm beeping or a light lighting up. Pressure sensors may also be located in areas of the cuff that include compression cavities. These pressure sensors monitor the amount of pressure that is being applied to the areas that are meant for pressure to be applied. Once again, if the pressure exceeds a certain threshold or is too low, an alarm or indication may be generated that notifies the patient. The control unit may also be capable of adjusting the flow rate of fluid being supplied by the fluid pump to compensate for either to much pressure or not enough pressure by increasing or decreasing the flow rate of the fluid.

The thermal compression system disclosed herein is economical and provides many advantages over current thermal compression systems. The use of a fluid pump to replace fluid in a fluid cuff thereby eliminating the need for much of the human interaction with the cuff provides many advantages. As set forth above, fluid is pumped from the fluid container to the cuff by the fluid pump and returned to the fluid container from the cuff in a closed loop fluid system. As such, after the cuff is applied to the portion of the body to be treated, the patient no longer needs to interact with the thermal compression system until more ice is needed in the fluid container. However, the amount of time that the fluid container is able to maintain the fluid at a cool enough temperature to provide adequate therapy will likely extend beyond the time period in which the patient needs to use the thermal compression system.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWING

FIG. 1 illustrates an exemplary thermal compression system of the present invention.

FIG. 2 illustrates a top view of a lower fluid cavity that forms a portion of a cuff of the thermal compression system.

FIG. 3 illustrates a top view of an upper fluid cavity that forms a portion of the cuff of the thermal compression system.

FIG. 4 illustrates a cross section view of the lower and upper sections of the cuff illustrated in FIGS. 2 and 3 at cross section A.

FIG. 5 illustrates a cross section view of the lower and upper sections of the cuff illustrated in FIGS. 2 and 3 at cross section B.

FIG. 6 illustrates a cross section view of the lower and upper sections of the cuff illustrated in FIGS. 2 and 3 at cross section A showing a pressure sensor and a temperature sensor.

### DETAILED DESCRIPTION OF THE PRESENTLY PREFERRED EMBODIMENTS OF THE INVENTION

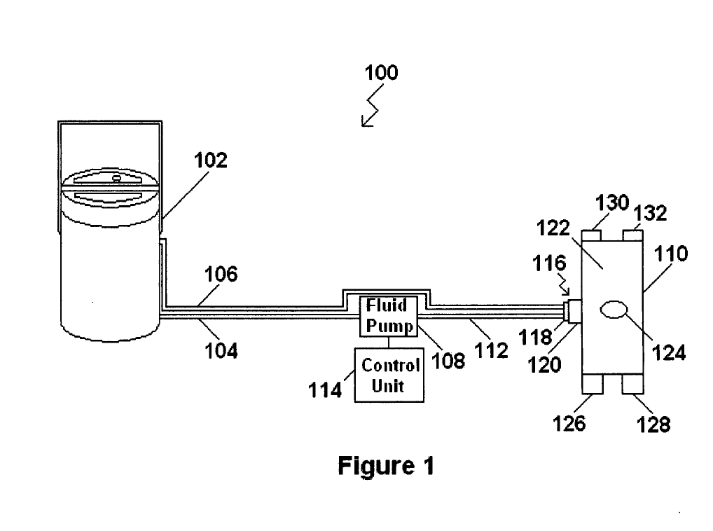

Referring to FIG. 1, a thermal compression system 100 is illustrated that is used to apply hot or cold fluid in combination with compression to a portion of a limb of a human body or an animal body. While the thermal compression system 100 disclosed herein may be used with hot or cold fluid, a majority of the description set forth below will focus on the use of cold fluid where its greatest use is anticipated. The thermal compression system 100 illustrated in FIG. 1 is formed to treat a knee of an individual. However, it should also be recognized that other areas of the body may be treated using one of several thermal compression systems 100 that may be formed in various different shapes designed to accommodate different areas of the human body.

The thermal compression system 100 may include a fluid container 102 that may be used to hold cold or hot fluid. In the case of cold fluid, the fluid container 102 may be filled with ice and water or any other suitable fluid that may be used to apply cold therapy to a portion of the body. The desired temperature of the cold fluid may be set to a predetermined level of coldness to treat the area of interest. Although not illustrated, in the case of hot fluid being used, the fluid container may include a heating system that heats the fluid to a predetermined temperature. The heating system may be an electronic coil-based heating system or any other suitable type of heating system.

The thermal compression system 100 may include a fluid output line 104 and a fluid input line 106 that are connected with the fluid container 102. These lines 104, 106 are used to allow fluid to exit and enter the fluid container 102. The fluid output line 104 may be positioned near the lower portion of the fluid container 102. The fluid input line 106 may be positioned near the upper portion of the fluid container 102. The fluid input line 106 may be positioned near the upper portion of the fluid container 102 so that fluid entering the fluid container 102 may travel through the cooling mechanism (e.g.—ice) or through the heating system to once again ultimately exit the fluid container 102 via the fluid output line 104. As such, this allows the fluid to become colder or hotter as it travels downward through the fluid container 102 until it ultimately exits the fluid container through the fluid output line 104. The size of the fluid output and input lines 104, 106 may vary as a function of the volume of fluid flow that needs to be provided for each particular type of limb that the thermal compression system 100 is being used to treat.

As further illustrated in FIG. 1, the thermal compression system 100 may also include a fluid pump 108 that may be connected with the fluid output line 104 and a cuff 110. The fluid pump 108 pumps fluid from the fluid container 102 into the cuff 110 from a pump output line 112 that is connected with the cuff 110. The fluid pump 108 may operate continuously to constantly supply the cuff 110 with a fresh supply of hot or cold fluid from the fluid container 102. The volume or amount of fluid that the fluid pump 108 is designed to pump per minute may vary depending on the specific design of the thermal compression system 100. The force at which the fluid pump 108 is capable of pumping fluid may also vary from application to application.

Although not specifically illustrated, the fluid pump 108 may include a timing circuit that turns the fluid pump 108 on and off at a predetermined time interval. For example, the fluid pump 108 may be turned on for fifteen-seconds every five minutes by the timing circuit to allow the fluid in the cuff 110 to be completely replaced with new fluid from the fluid container 102 once every five minutes. Different time intervals may be used depending on the design of the thermal compression system 100. The fluid pump 108 may be powered by batteries, a common wall outlet or any other suitable power source.

As further illustrated in FIG. 1, the thermal compression system 100 may also include a control unit 114. The control unit 114 may be connected with the fluid pump 108 and may be used to control operation of the fluid pump 108. The fluid pump 108 may be capable of pumping fluid at variable flow rates. The control unit 114 may be used to control the flow rate of the fluid pump 108. The control unit 114 may also be capable of turning on and off the fluid pump 108 at predetermined time intervals, thereby eliminating the need for the timing circuit described above. The control unit 114 may be used to control the overall operation of the fluid pump 108 as well as the thermal compression system 100, as set forth in greater detail below.

The thermal compression system 100 may also include a connection device 116 that may be used to connect the pump output line 112 and the fluid input line 106 with the cuff 110. The connection device 116 may include at least one male engagement member 118 and a female engagement member 120. The connection device 116 may allow the pump output line 112 and the fluid input line 106 to be removably connected with the cuff 110. The female engagement member 120 may be connected with the cuff 110 and the male engagement member 118 may be connected with the pump output line 112 and the fluid input line 106. The male engagement member 118 and the female engagement member 120 may also be reversed in other examples of the invention.

The connection device 116 is preferably a quick disconnect device that allows the male and female engagement members 118, 120 to be easily connected and disconnected with one another in a manner that provides a fluid tight connection. In other examples of the invention, the thermal compression system 100 may not include a connection device 116 and the pump output line 112 and the fluid input line 106 may directly be connected with the cuff 110.

Referring to FIG. 1, the cuff 110 is a uniform body 122 that is preferentially substantially formed from a flexible fluid impervious material. The uniform body 122 may include an opening 124 that is intended to receive or be placed over the patella or kneecap of the knee or various other body parts. The opening 124 in the uniform body 122 does not allow thermal therapy or pressure to be applied to the patella of the person wearing the thermal compression system 100. The patella of the knee does not normally need to receive thermal compression during thermal therapy applications.

The cuff 110 may also include a first proximal connection mechanism 126 that is connected with the cuff 110 at a predetermined side of the cuff 110. The first proximal connection mechanism 126 may be made from a stretchable material or any other type of suitable material. The cuff 110 may also include a first distal connection mechanism 128 that is similar to the first proximal connection mechanism 126. In addition, the cuff 110 may also include a second proximal connection mechanism 130 and a second distal connection mechanism 132 that may be located at an opposite side of the first proximal connection mechanism 126 and the first distal connection mechanism 128. The second proximal and distal connection mechanisms 130, 132 may also be made from a stretchable material or any other type of suitable material. The connection mechanisms 126-130 may also be formed on or as a part of the uniform body 122.

The proximal connection mechanisms 126, 130 and the distal connection mechanisms 128, 132 may be used to connect the cuff 110 to the knee of the body or to other body parts in other applications of the invention. The connection mechanism used may be Velcro or any other suitable mechanism that may be used to connect objects together. The connection mechanisms 126-130 allow the cuff 110 to be secured around or to the part of the body that needs to be treated by the thermal compression system 100. In the discussion of the figures that follow, like numbered elements refer to the same elements throughout the various figures.

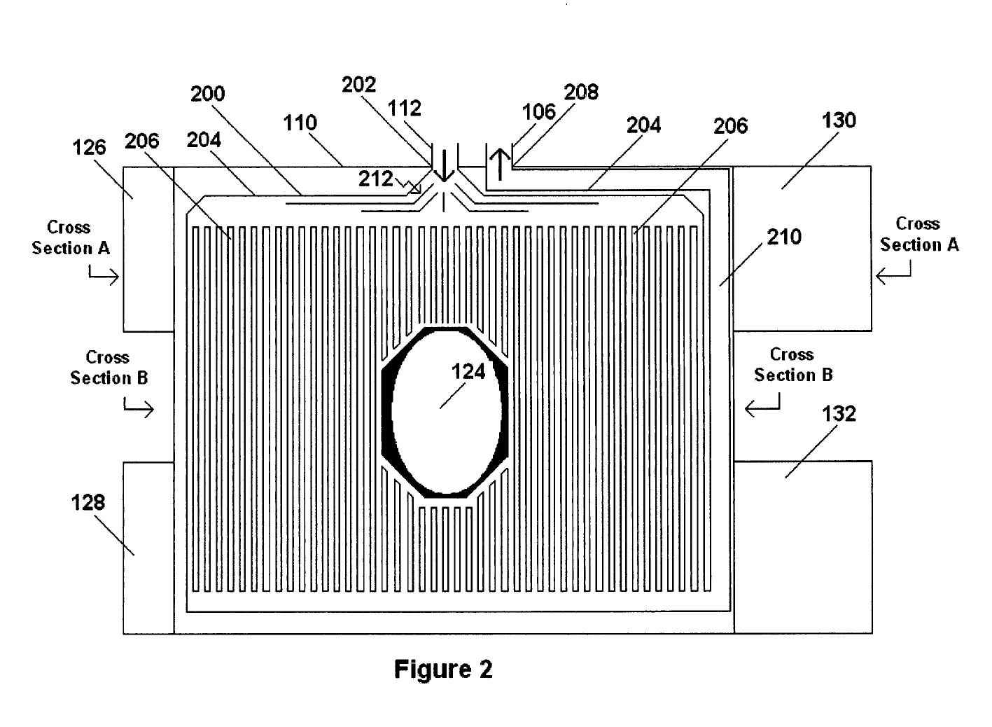

Referring to FIG. 2, a top view of a lower fluid section 200 that forms a portion of the cuff 110 of the thermal compression system 100 is illustrated. The lower fluid section 200 may include a cuff fluid input opening 202 that allows the pump output line 112 to pump fluid into the lower fluid section 200. The lower fluid section 200 may also include at least one fluid receiving chamber 204 that may be designed in a predetermined shape to receive fluid that is supplied by the pump 108. The fluid receiving chamber 204 is designed in a manner that allows fluid to flow optimally throughout the fluid receiving chamber 204.

During treatment, the fluid receiving chamber 204 fills up with fluid that is supplied from the fluid container 102 via operation of the fluid pump 108. The lower fluid section 200 may also include at least one fluid restriction chamber or cavity 206 that may not allow fluid from the pump output line 112 to enter particular areas of the fluid receiving chamber 204. The lower fluid section 200 may include a cuff fluid output opening 208 that allows the fluid input line 106 to receive fluid that needs to be transferred back to the fluid container 102. The fluid receiving chamber 204 may have a fluid output chamber or cavity 210 that may be used to discharge fluid out of the fluid receiving chamber 204 into the fluid input line 106, where it ultimately is directed back into the fluid container 102. In addition, the fluid receiving chamber 204 may include a directional fluid dispenser 212 that directs fluid towards different areas of the fluid receiving chamber 204 so that the entire fluid receiving chamber 204 becomes filled with fluid.

The fluid receiving chamber 204 may be designed in any shape to apply thermal therapy to an area of interest of the body. The design illustrated in FIG. 2 should be viewed in an illustrative sense only and not as a restriction of the present invention unless otherwise claimed. In addition, the fluid restriction chamber 206 may be designed in any shape that is preferential for the area of interest of the body. In particular, the fluid restriction chambers 206 may be designed to be generally located where blood flow paths are located in the portion of the body being treated thereby minimizing blood flow restriction in those important areas. The fluid receiving chamber 204 and the fluid restriction chamber 206 are preferentially designed to hold enough volume of fluid so as to not apply enough pressure on the portion of the body to be treated to restrict blood flow in the area being treated. As such, in some embodiments, the fluid restriction chamber 206 may not even be used as the fluid receiving chamber 204 may be designed to hold a volume of fluid that will not restrict the blood flow in the area of interest.

Although not specifically illustrated in FIG. 2, the underside of the lower fluid section 200 is the portion of the cuff 110 that comes into contact with the portion of the limb of the body that is receiving therapy. In other words, the lower fluid cavity 200 is used to apply or transfer cold or warmth to the area of interest of the body. The connection mechanisms 126-132 allow the cuff 110 to be attached to the portion of the body that is to be treated. As such, in the example illustrated in FIG. 2, the cuff 110 may be completely wrapped around the knee of a human and the entire knee, except for the portion of the knee located at the opening 124, may receive cold or warm therapy by the cuff 110. As set forth in greater detail below, preferentially the cuff 110 is not secured to the area of the body being treated in a tight manner, but in a manner only tight enough to allow the underside of the lower fluid section 200 to come into contact with the area of the body being treated using the cuff 110.

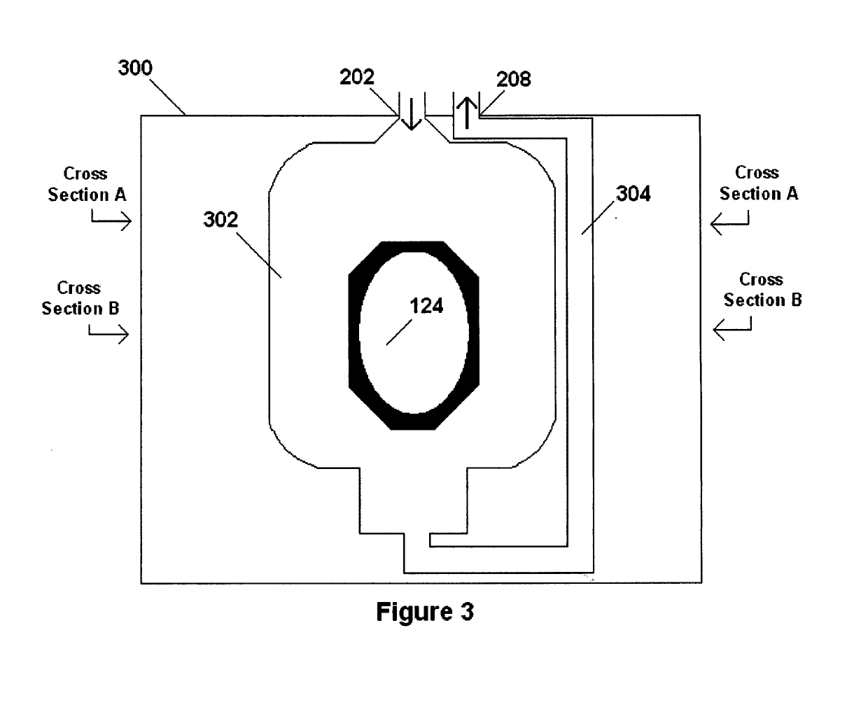

Referring to FIG. 3, a top view of an upper fluid section 300 that may form a portion of the cuff 110 of the thermal compression system 100 is illustrated. The upper fluid section 300 may be connected with the lower fluid section 200. The upper fluid section 300 may be connected with an upper side of the lower fluid section 200, thereby leaving the underside of the lower fluid section 200 exposed so that it may be used to treat the portion of the body where it is designed for use. As set forth in detail below, the upper fluid section 300 is used to apply or generate a predetermined amount of compression or pressure in predetermined areas of the cuff 110, which allows compression or pressure to be applied to the lower fluid section 200 and in turn, to the portion of the body being treated.

The upper fluid section 300 may include the cuff fluid input opening 202 and the cuff fluid output opening 208. The upper fluid section 300 may also include its own openings that are separate from the cuff fluid input opening 202 and the cuff fluid output opening 208. A compression chamber 302 may be included that is connected with the pump output line 112 and may be positioned near the cuff fluid input opening 202. As such, during operation the compression chamber 302 receives fluid from the pump 108 via the pump output line 112. The pump output line 112 may be connected with the compression chamber 302 through the cuff fluid input opening 202. If the connection device 116 is used in the cuff 110, the pump output line 112 and the compression chamber 302 may be connected with the connection device 116.

The compression chamber 302 may also include a compression chamber output line or cavity 304 that is used to return fluid to the container fluid 102. The compression chamber output line 304 may be connected with the fluid input line 106 of the container 102. The compression chamber output line 304 may extend beyond the outside perimeter of the cuff 110 or may be in the inside of the cuff 110, which is true of all fluid lines of the cuff. During operation, fluid is pumped from the fluid container 102 by the fluid pump 108 into the compression chamber 302, thereby filling the compression chamber 302, and then exits the compression chamber 302 through the compression chamber output line 304. Filling the compression chamber 302 with fluid causes fluid to exit the compression chamber 302 through the compression chamber output line 304 once it has expanded to a predetermined expansion point.

The compression chamber 302 is shaped in a manner to apply pressure to areas of the limb of the body where it is needed without restricting blood flow to the limb. As such, the compression chamber 302 is formed in a special non-blood flow restriction shape that is dependent upon the particular limb or portion of the body that the thermal compression system 100 is designed to treat. As well known in the medical field, each limb or portion of the body contains veins or arteries that carry blood to the limb or portion of the body as well as on to other places in the body.

The compression chamber 302 creates a watertight cavity that, in the case of a knee being treated, is shaped to preferentially to envelope the anterior and sides of the knee as well as the suprapatellar pouch. These are the areas where post-trauma body fluids typically accumulate and where cold and compression are most needed in the knee. The cuff 110 is economically fabricated from sheets of material and permits adjustable shaping so as to conform to the knee even when the knee and cuff are flexed at different angles.

The expansion of the cuff 110 from inflation becomes greater in the area above the patella (where swelling is greatest) and the expansion is restricted in the area below the patella (where swelling is less). It is well known medically and tests demonstrate that venous flow is far more sensitive to constriction in the lower underside portion of the knee and less sensitive in the upper underside portion of the knee. Thus, by limiting tightening of the distal connection mechanisms 128, 132 and by designing the compression chamber 302 to avoid these areas, little or no pressure is applied below the knee or in back of the knee and constriction of venous flow is minimized or almost avoided altogether.

Although not illustrated in FIGS. 2 and 3, if the connection device 116 is used with the cuff 110, at least a portion of the connection device 116 will be connected with the cuff 110. The connection device 116 will likely have a pair of connectors that may be connected with the fluid receiving cavity 204 and the fluid output cavity 210. The compression chamber output line 304 may be connected with or formed as a part of the fluid output cavity 210. If the compression chamber output line 304 is not formed separately or connected with the fluid output cavity 210, it may be connected with the connection device 116, which allows the fluid to exit the compression chamber 302.

The compression chamber 302 supplies compression to the desired areas being treated in at least two different ways. First, as the compression chamber 302 inflates when fluid is pumped into the compression chamber 302, pressure is applied to the desired areas because the connection mechanisms 126-132 hold the cuff 110 in place snug against the portion of the body being treated. This creates some compression against the body.

In addition, fluid pressure equations may be used to calculate the amount of pressure that is applied when fluid is pumped into the compression chamber 302. Generally speaking, the pressure of the fluid in the compression chamber 302 may be calculated using the following equation: P=ρgh. Where ρ represents the density of the fluid, g represents the acceleration of gravity, and h represents the depth of the fluid. As such, the compression chamber 302 may be designed to expand to various heights to achieve a predetermined amount of pressure in the particular area of the body being treated. Additional equations and formulas may be used to calculate the pressure that is supplied so that the thermal compression system 100 does not apply too much pressure in areas of the body.

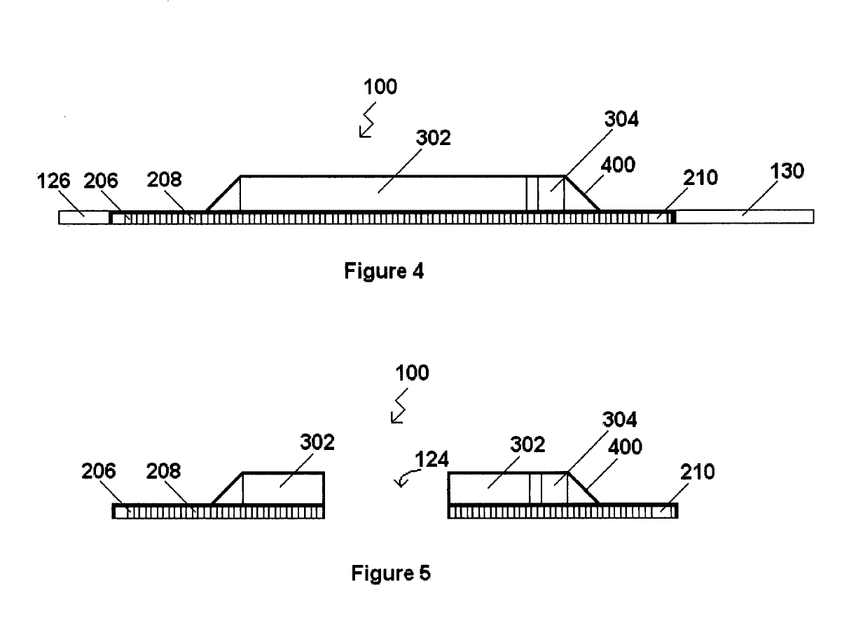

Referring to FIG. 4, a cross-sectional view of the cuff 110 is illustrated that depicts the lower fluid section 200 connected with the upper fluid section 300 at Cross Section A in FIGS. 2 and 3. A fluid height restriction member 400 may be connected with a top surface of the compression chamber 302 and a top surface of the lower fluid section 200. The fluid height restriction member 400 may limit the height to which the compression chamber 302 may expand when filled with fluid. This will also help cause pressure to be applied to the top surface of the lower fluid section 200, thereby applying pressure to the area of the body being treated where the compression chamber 302 is located.

As illustrated in FIG. 4, the compression chamber 302 is positioned on top of the lower fluid section 200 at predetermined locations where blood flow will not be restricted by use of the cuff 110. The compression chamber output line 304 is also illustrated next to the compression chamber 302. The first and second proximal connection mechanisms 126, 130 of the lower fluid section 200 are also illustrated. In addition, the fluid receiving chamber 204, the fluid restriction chamber 206 and the fluid output line 210 are clearly illustrated. FIG. 4 illustrates the lower fluid section 200 in an inflated state and it should be recognized that in a deflated state the lower fluid section 200 may look different since the lower fluid section 200 is preferentially made using a substantially flexible fluid impervious material that will stretch into a predetermined shape when fluid is introduced into the areas where fluid is meant to be introduced.

Referring to FIG. 5, a cross-sectional view of the cuff 110 is illustrated that depicts the lower fluid section 200 connected with the upper fluid section 300 at Cross Section B in FIGS. 2 and 3. The opening 124 of the cuff 110 that exposes the patella of the knee is illustrated in FIG. 5. The first and second proximal connection mechanisms 126, 130 are not illustrated in this figure. Other than these changes, FIG. 5 is substantially similar to FIG. 4 and as such, a detailed discussion of FIG. 5 is not necessary.

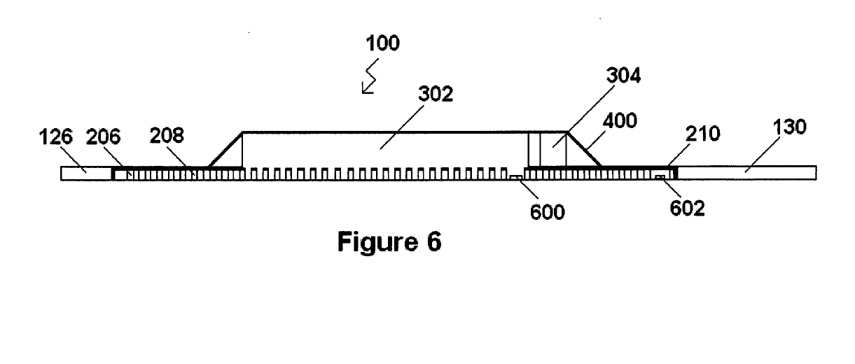

Referring to FIG. 6, the cuff 110 may also include a pressure sensor 600 and a temperature sensor 602. The pressure sensor 600 and the temperature sensor 602 may be connected with the control unit 114. The pressure sensor 600 may be used to monitor the pressure that is applied to the portion of the body that is being treated. If the pressure becomes to high, an alarm may be generated that informs the patient that the cuff 110 needs to be removed. The pressure sensor 600 may also be located in areas other than areas where the compression chamber 302 is located. For example, pressure sensors 600 may be placed in the areas where it is desirable to limit the pressure being applied to the portion of the body to limit blood flow restriction. If the fluid pump 108 is a variable flow rate pump, the control unit 114 may also be able to adjust the flow rate of the fluid pump 108 to lower the pressure or raise the pressure to a desired level.

The temperature sensor 602 may be used to monitor the temperature of the fluid as it exits the cuff 110. In addition, temperature sensors 602 may be placed throughout the cuff to monitor the temperature of the fluid being used to supply the thermal therapy to the portion of the body being treated. If the temperature of the fluid being used to treat the portion of the body becomes to low, an indication may be generated that notifies that person using the thermal compression system 100. In addition, if the fluid being used to treat the patient becomes either to warm or cold in the cuff 10, the control unit 114 will be notified of this fact and can turn on the fluid pump 108 to provide a fresh supply of fluid from the fluid container 102.

FIG. 6 also illustrates an embodiment of the invention in which the compression chamber 302 is integrated as part of the fluid receiving chamber 204. As such, this would eliminate the need for manufacturing an upper and lower fluid section 200, 300. The compression chamber 302 would be used in areas of the cuff 110 where compression was desired to be applied to the portion of the body being treated. Areas of the cuff 110 that cover locations of the portion of the body that contain a blood flow path of interest would not include a compression chamber 302.

The present invention discloses a thermal compression system 100 that may be used to treat various portions of the human body. The thermal compression system 100 includes a cuff 110 that may be wrapped around the portion of the body that needs to be treated. The connection mechanisms 126-132 are used to secure the cuff 110 to the portion of the body being treated. Preferentially, the cuff 110 is attached to the portion of the body in a deflated or unfilled state and is not attached to the limb tight, but in a manner that allows the cuff to loosely come into contact with the area of the body to be treated. In other words, when the cuff 110 is attached to the portion of the body that is being treated, the cuff 110 is wrapped around the portion of the body to be treated such that the cuff 110 makes light contact with the skin.

After the cuff 110 is attached to the portion of the body to be treated, the fluid pump 108 is activated thereby causing fluid from the fluid container 102 to be pumped into the cuff 110. The cuff 110 then fills with fluid, and once filled, fluid may begin to exit the cuff 110 and return to the fluid container 102. If the pump 108 remains active at all times, fluid will continuously travel from the fluid container 102 to the cuff 110 and back from the cuff 110 to the fluid container 102 thereby forming a closed loop fluid path. If a timing system is used, fluid will fill the cuff 110 and after a predetermined amount of time, the fluid in the cuff 110 will be replaced with freshly chilled or heated fluid from the fluid container 102.

The present invention solves the problems with the prior art by eliminating the need for the patient to manually change ice packs or manually lift the container up and down to fill the cuff with fluid. The present invention also provides a cuff 110 that provides optimal coverage of the portion of the body being treated. In addition, the present invention provides a cuff 110 that provides optimal compression in areas where it is needed while minimizing compression in areas that may restrict blood flow.

While various embodiments of the invention have been described, it will be apparent to those of ordinary skill in the art that many more embodiments and implementations are possible that are within the scope of the invention. Accordingly, the invention is not to be restricted except in light of the attached claims and their equivalents.

1. A thermal compression system adapted to encompass a portion of a limb, comprising:
a fluid container having a fluid output line and a fluid input line;
a fluid pump connected with the fluid output line of the fluid container;
a cuff connected with an output of the fluid pump that receives fluid from the fluid container through the fluid pump;
where the cuff includes at least one fluid chamber that receives fluid from the fluid pump, where the at least one fluid chamber is formed having a predetermined shape that avoids applying compression in at least one area of the limb that has at least one blood flow path while at the same time applies compression in at least one therapeutic area of the limb; and where the at least one fluid chamber includes at least one cuff output that is connected with the fluid input line of the fluid container that allows fluid to be returned to the fluid container from the cuff.

2. The thermal compression system of claim 1, further including a control unit connected with the fluid pump that controls operation of the fluid pump.

3. The thermal compression system of claim 2, where the control unit includes a timer that runs the fluid pump at predetermined time intervals to provide fluid from the fluid container to the cuff while at the same time returning fluid from the cuff to the fluid container.

4. The thermal compression system of claim 1, where the fluid pump runs continuously to constantly provide fluid from the fluid container to the cuff and return fluid from the cuff to the fluid container.

5. The thermal compression system of claim 1, further comprising a control unit connected with the fluid pump and a temperature sensor that is located in the fluid chamber, where the control unit turns the fluid pump on when the temperature sensor indicates that the fluid in the cuff reaches a predetermined temperature threshold.

6. The thermal compression system of claim 1, further comprising a control unit connected with the fluid pump and a pressure sensor that is located in the fluid chamber, where the control unit generates an indication if the pressure sensor senses that pressure being applied by the cuff reaches a predetermined threshold.

7. The thermal compression system of claim 1, further comprising at least one quick-disconnect connector that connects the output line of the fluid pump and the fluid input line of the fluid container with the cuff.

8. A thermal compression system, comprising:
means for securing a cuff to an outside portion of a limb;
means for holding fluid;
a fluid pump for pumping fluid into the cuff from the means for holding fluid;
means for removing fluid from the cuff and returning fluid to the means for holding fluid; and
where the cuff includes means for restricting compression being applied to at least one blood flow path of the limb while at the same time providing compression in at least one therapeutic area of the limb.

9. A method of applying thermal compression to part of a human body, comprising the steps of:
(a) securing a cuff to engage an outside portion of a limb;
(b) supplying fluid to the cuff with an electric fluid pump;
(c) restricting compression being applied by the cuff to an outside portion of the limb in an area containing at least one blood flow path;
(d) supplying compression to the outside portion of the limb in at least one therapeutic area; and
(e) removing fluid from the cuff while at the same time replacing the removed fluid with new fluid.

10. The method of claim 9, where the cuff is secured to the outside portion of the limb using a connection mechanism that includes a pair of Velcro straps.

11. The method of claim 9, where the electric fluid pump is connected with a fluid container that holds fluid for use with the cuff.

12. The method of claim 9, where the step of restricting compression is accomplished by prohibiting fluid from entering a compression chamber of the cuff that is located in the area of the at least one blood flow path.

13. The method of claim 9, where the step of supplying compression is accomplished by filling a compression chamber of the cuff with fluid located in the at least one therapeutic area of the limb.

14. The method of claim 9, where fluid is removed from the cuff through a cuff output and returned to a fluid container.

15. A thermal compression system adapted to encompass a portion of a limb, comprising:
a fluid container having a fluid output line and a fluid input line;
a fluid pump connected with the fluid output line of the fluid container;
a cuff connected with the fluid pump;
where the cuff includes a lower fluid chamber that receives fluid from the fluid pump, where the cuff includes an upper fluid chamber that receives fluid from the fluid pump, where the upper fluid chamber is formed in a predetermined shape that avoids applying compression in at least one area of the limb that contains at least one blood flow path while at the same time supplying compression in at least one area of the limb that requires therapy, and where the lower fluid chamber and the upper fluid chamber include at least one output that is connected with the fluid input line of the fluid container that returns fluid to the fluid container.

16. The thermal compression system of claim 15, further including a control unit connected with the fluid pump that controls operation of the fluid pump.

17. The thermal compression system of claim 16, where the control unit includes a timer that runs the fluid pump at predetermined time intervals to circulate the fluid from the fluid container through the cuff and back to the fluid container for a predetermined amount of time.

18. The thermal compression system of claim 15, where the pump runs continuously thereby supplying fluid from the fluid container to the cuff while at the same time returning fluid from the cuff to the fluid container.

19. The thermal compression system of claim 15, further comprising a control unit connected with the pump and a temperature sensor that is located in the lower fluid cavity of the cuff, where the control unit turns on the pump when a temperature of the fluid rises above a predetermined level.

20. The thermal compression system of claim 15, further comprising a control unit connected with a pressure sensor located in the upper fluid cavity that monitors the pressure being applied to the portion of the limb.

21. The thermal compression system of claim 15, where the upper fluid chamber is formed to hold more fluid in areas where more compression is desired and less fluid in areas where less compression is desired.

22. A thermal compression system adapted to encompass a knee of a person, comprising:
a container having a fluid output line and a fluid input line, where the container holds fluid used for treating the knee;
a cuff including a fluid input, a fluid output, and a fluid chamber that includes at least one fluid receiving cavity and at least one compression cavity, where the cuff is capable of being secured around the knee so that a surface of the cuff is in contact with the knee;
a fluid pump connected with the fluid output line of the container and the fluid input of the cuff, where the fluid pump supplies fluid to the fluid cavity and the compression cavity; and
where the fluid receiving cavity supplies thermal therapy to the knee and the compression cavity applies pressure to at least one area of the knee that does not contain a blood flow path, where the fluid output of the cuff is connected with the fluid input line of the container, where fluid exits the cuff through the fluid output thereby being returned to the container.

23. The thermal compression system of claim 22, further comprising an opening in the cuff that is used to position the patella of the knee in the cuff.

24. The thermal compression system of claim 22, further including a control unit connected with the fluid pump that controls operation of the fluid pump.

25. The thermal compression system of claim 24, where the control unit includes a timer that runs the fluid pump at predetermined time intervals to provide fluid from the fluid container to the cuff while at the same time returning fluid from the cuff to the fluid container.

26. The thermal compression system of claim 22, where the fluid pump runs continuously thereby constantly providing fluid from the fluid container to the cuff and returning fluid from the cuff to the fluid container.

27. The thermal compression system of claim 22, further comprising a control unit connected with the fluid pump and a temperature sensor that is located in the fluid chamber, where the control unit turns on the fluid pump when the temperature sensor indicates that the fluid in the cuff reaches a predetermined temperature.

28. The thermal compression system of claim 22, further comprising a control unit connected with the fluid pump and a pressure sensor that is located in the fluid chamber, where the control unit generates an indication if the pressure sensor indicates that a pressure being applied by the cuff reaches a predetermined level.
